(19) United States
(12) Patent Application Publication
(10) Pub. No.: US 2023/0191169 A1
(43) Pub. Date: Jun. 22, 2023

(54) HEADPIECE, FACE SHIELD AND METHOD OF USING A HEADPIECE

(71) Applicant: Sonke Mestemacher, Fehmarn (DE)

(72) Inventor: Sonke Mestemacher, Fehmarn (DE)

(21) Appl. No.: 17/927,153

(22) PCT Filed: Apr. 8, 2021
(86) PCT No.: PCT/DE2021/000067

§ 371 Date: Nov. 22, 2022

(30) Foreign Application Priority Data
May 22, 2020 (DE) DE102020003054.8
Jun. 8, 2020 (DE) DE102020003448.9
Sep. 9, 2020 (DE) DE102020005503.6

(51) Int. Cl. A62B 18/00; A41D 13/11; A61L 9/20; A61L 9/12; A62B 18/08; A62B 23/02

(52) U.S. Cl. CPC A62B 18/003; A41D 13/1184; A41D 13/1161; A61L 9/20; A61L 9/122; A62B 18/006; A62B 18/08; A62B 23/02; A61L 2209/12; A61L 2209/14; A61L 2209/111

(57) ABSTRACT

A headpiece (1) worn by a user provides air to the user from which airborne disease particles have been removed. The headpiece includes an air guide (2) through which air flows due to fans (13, 14). Antibacterial and/or antiviral devices (3) such as filters and ultraviolet (UV) disinfecting lights operate as air purifiers to purify air that passes through the air guide. The purified air is delivered to an area between the inside surface of a visor (10) and a face (11) of the person wearing the headpiece.

### TECHNICAL FIELD

Exemplary arrangements relate to a headpiece. A headpiece may be any device that enables an object to be held on the head of a user. In some arrangements this can be a headband, which can support an object such as a headlamp. It can also be a hat, a cap, a mask, or a face visor. A headpiece can also be attached to a person's throat, or to the nape of the neck. Such holders are sometimes known as neck pillows, or as holders for microphones. In particular exemplary arrangements a headpiece is a holder that enables an object to be positioned within a region of less than 20 cm from the mouth-nose region. Exemplary arrangements further relate to a face shield, and to a method of using a face shield, and in particular a headpiece. Further exemplary arrangements relate to a visual shield. A visor, visual shield or face shield for purposes hereof shall include any device that protects the face, eyes, mouth, nose, skin and, depending on the design, also the ears, and through which one can see. The protection may be with respect to all solid, liquid and gaseous objects emanating from the external environment, as well as to radiation of all kinds.

### BACKGROUND

Due to the Covid-19 pandemic, it has become clear that personal protection against microorganisms such as viruses, bacteria, germs, molds, etc. (collectively referred to as airborne disease particles herein) is becoming increasingly important, both today and in the future.

People are prepared to accept restrictions and expenditures in order to continue to participate in public life, as evidenced by the wearing of masks. At the same time, there is an economic factor that should not be underestimated when people succeed in gathering together more closely again at events and in public spaces. Events of all kinds can then take place again without restrictions.

With the present mouth-nose coverings, or Perspex™ shields in front of the face, together with appropriate distancing rules, everyone is protected as well as possible from others. Self-protection is only possible to a limited extent with these measures.

Protective equipment such as that worn in laboratories and hospitals is not really practical for constant use in private or public spaces because of the effort and cost involved.

Protective equipment and methods to prevent the spread of airborne diseases may benefit from improvements.

### SUMMARY

Exemplary arrangements relate to a headpiece. The exemplary headpiece ensures that the person using this headpiece always inhales air and comes into facial contact with such air, in which the viruses and bacteria etc. and other airborne disease particles have previously been killed or at least decimated or substantially reduced.

An exemplary headpiece with an air guide, which has an antibacterial and/or antiviral device, consists essentially of a holder for the attachment of a mobile device such as a visual shield in the region of a person's head, and an antibacterial and/or antiviral device. In some exemplary arrangements, the air guide enables open air to be received from the environment and released in the region of the face of the wearer. Such an air guide may in turn have a conduit and/or a protective shield that directs air towards the mouth-nose region of a person wearing the device.

While current caps may be uncomfortable to wear if they have an antiviral device, the headpiece of the exemplary arrangements is easy to put on, easy to wear and also easy to take off. It can be slipped onto the face, and all required components of the antibacterial and/or antiviral device can be integrated into the headband. A top of head covering may be optional, while a visor in some arrangements is useful.

In order to provide the wearer of the headpiece with sufficient space to wear visual aids, such as glasses, and in order to maintain a storage volume of purified air in an area between the wearer's face and the visor, a particular shape of the visor is used in exemplary arrangements. This visor shape can be configured to also ensure that the flow velocity at the edges is increased by keeping the air gap at the edges as small as possible so as to make it more difficult for contaminated air to penetrate against the flow.

For this purpose, in some arrangements the angle between a front face of the visor and the headband is greater than the angle between a side face and the headband. It may be useful in some arrangements if the visor is arranged on a front face at an angle of about 85° to the central plane of the headband. Thus the visor does not extend downwards from the central plane of the headband at an angle of 90°, but is slightly inclined inwards towards the face. To ensure that the visor is disposed close to the head, in some arrangements the visor is arranged at an angle of about 75° to the central plane of the headband on each side of a user's face. On each side also, the visor thus does not extend downwards from the plane of the headband at an angle of 90°, but is slightly inclined inwards towards the user's head. For purposes of these angles the term about is defined as within +/−2°.

The particular shape of the visor in some exemplary arrangements also results in less reflection from light sources located above the visor, such as ceiling lamps. Such reflections can also be minimized by the selection of appropriate less reflective materials, and an anti-reflection coating on the visor's field of vision on its inner face or on its outer face.

The headpiece of exemplary arrangements thus makes it possible to supply disinfected, or antibacterially and/or antivirally treated air to a person, which the person can breathe in immediately. The air guide of exemplary arrangements is configured such that the person breathes in exclusively, or at least predominantly, air that has been treated.

Exemplary arrangements allow the person wearing the headpiece to move freely in society, and, through the use of the special headpiece, to have a minimum of personal restriction, while at the same time having maximum personal protection from the microorganisms in the ambient air, together with the aerosols in the air contaminated by the latter.

The headpiece of exemplary arrangements can be combined with various other devices. For example, some exemplary headpieces can additionally have a light, a camera or a microphone. In some headpiece arrangements the headpiece may have a head covering which extends across the top of a user's head. In some arrangements a head covering provides protection for the person and can give the headpiece sufficient support to be worn securely on or against the head. However, in some arrangements devices such as headlamps or microphone holders may also be combined with an air guide, which has an antibacterial and/or antiviral device, so as to provide a suitable headpiece.

In some exemplary arrangements in which the headpiece has an attached transparent visor, this can then protect the person wearing the headpiece, as the visor can protect against droplet and aerosol airborne diseases. In addition, a visor can help to guide the air. For example, in an area between the head of a person wearing the headpiece and the inside surface of the visor, antibacterially and/or antivirally treated air can be fed to the person's mouth-nose region by directing the air between the visor and the face. The visor then forms at least part of the air guide.

In an exemplary arrangement provision is made for the air guide to have a fan. This fan can blow or suck the air through the antibacterial and/or antiviral device. As used herein a “fan” is understood to mean any type of fan, ventilator or pump for the movement or supply of air or gases, in particular including rotor-less fans, as in the Dyson™ Air Multiplier, for example.

In exemplary arrangements, the antibacterial and/or antiviral device can, for example, include a filter or an antibacterial or antiviral active surface. In some arrangements the filter may comprise an electrostatic filter. In some arrangements the filter may also use chemically or biologically active additives. In exemplary arrangements the antibacterial and or antiviral device may include an ultraviolet (UV) light, and in particular a UVC light. This makes it possible to treat air with a special lamp so as to achieve an antibacterial or antiviral effect.

In exemplary arrangements the antibacterial and/or antiviral device has at least one UV lamp, a UVC light source or UVC luminaire. The exemplary light source includes an encapsulated UVC light source. The exemplary light source, lamp, or luminaire, is configured such that its emitted radiation damages or kills microorganisms. In exemplary arrangements the UV lamp and the UVC lamp may include a lamp with LED technology.

In exemplary arrangements using a light source as an antibacterial and/or antiviral device, an energy source is utilized, which in such arrangements may be integrated into the headpiece. Rechargeable batteries, single-use batteries, or solar cells, may be used in some exemplary arrangements as suitable energy sources. Since the exemplary headpiece is worn directly on the body, energy can also be obtained from the temperature difference between the body temperature and the ambient temperature, for example for a UVC lamp. Thermal resonators or thermal electric generators (TEGs) may be used for this purpose.

In some exemplary arrangements a compact headpiece is made possible when the air guide directs the air through the antibacterial and/or antiviral device into an outflow region between the head and a visor. In this case, the visor serves as a protection against ambient air, and as a flow guide device for the antibacterially and/or antivirally treated air.

In some exemplary arrangements airflow from top to bottom in the areas bounded by the user's face and the inside surface of the visor is achieved if the airflow has an inflow region in a region above the outflow region. In this arrangement, the person's own exhaled breath can also be carried away with the supply of antibacterially and/or antivirally treated air.

Exemplary arrangements are utilized in accordance with a method of using a headpiece, in which air is fed through an antibacterial and/or antiviral device arranged on the headpiece to the wearer of the headpiece.

Exemplary arrangements may alternatively include a headpiece that is thus also a mobile unit with an antibacterial and/or antiviral device, with an airflow that can be directed towards a user's face, with or without a visor or face shield. Exemplary apparatus of this type can be attached to a person's head using various devices and structures (Velcro™, tape, an adhesive device, etc.).

In exemplary arrangements a headpiece may have a face shield that can be configured as a visor. In some arrangements the face shield can be positioned by different headpieces in front of the face of a person wearing the face shield. In some arrangements this can be from above by way of an independent holder, or also or alternatively a holder on the neck of a person. Such neck holders may include neck pillows, or holders for microphones. Combinations of the exemplary headpiece with head coverings such as hats, caps, helmets, etc. may be utilized in some arrangements.

In some arrangements a face shield, in combination with a disinfection and ventilation unit, ensures that the person using this face shield always breathes in disinfected air and the face comes into contact with such air in which the viruses and bacteria etc. have previously been killed, or at least decimated and substantially reduced. These exemplary disinfection and ventilation units can be in the form of electrostatic filters, UVC disinfection units, or other devices for killing viruses, bacteria or germs.

In some exemplary arrangements a ventilation outlet providing disinfected air may be provided on one, two, three, or four, edges of a face shield. This outlet can be connected to the disinfection and ventilation unit via an air guide. In some exemplary arrangements such an air guide can have a branch onto the surface of the face shield so as to cause disinfected air to flow in a targeted manner onto the mouth and/or nose region of the user. This results in the targeted supply of purified air to precisely this region when the user is breathing in. This can reduce the risk of aspiration and inhalation of secondary air that has not been purified by the disinfection and ventilation unit. Depending on the task in which the apparatus is used, these ventilation outlets for the purified air can be used all together, or individually, as well as in combination, to provide ventilation and air flow to the field of vision. In exemplary arrangements this brings a greater volume of air at a lower flow rate into the region between the face and the inside surface of the shield, making it more difficult for unpurified air to enter from the sides of the shield.

In addition, in some arrangements this also makes it possible to combine ventilation and exhaust. This provides active/passive protection, provided that not only the inhaled air but also the exhaled air is treated by means of appropriate disinfection units or filters. This is the case, for example, in an exemplary arrangement in which disinfected air is supplied from the top and/or transverse sides of the face and air is exhausted on the bottom edge.

Exemplary arrangements make it possible to protect not only oneself, but also the environment, when visiting old people's homes and other particularly sensitive facilities. This is achieved with a minimum of personal restriction and at the same time maximum personal protection, and protection of the environment, by reducing the contaminated aerosols released into the ambient air in the event that the wearer him or herself is ill.

The headpiece of exemplary arrangements can be combined with various other devices. For example, exemplary headpieces can additionally have a light, a camera or a microphone. Also possible are apertures for e.g. special devices such as visual aids for dentists. In such arrangements since the air gap between such special devices and the face shield also allows air to flow from the inside to the outside by virtue of the overpressure above atmospheric pressure in the area between the face and the face shield, the person wearing the device is thereby protected.

In such exemplary arrangements, the face shield can be supported by a headpiece, which also takes care of the supply of air and, if necessary, the disinfection and disposal of the air from the user.

In exemplary arrangements an arrangement of the air vents can be positioned in such a way that there is always an overpressure between the face and the shield compared to the air surrounding the head. This overpressure and the flow direction and velocity created by the positioning and arrangement of the outlet vents delivering disinfected air support this process, and thus make it more difficult for unpurified ambient air to access the region in front of the user's face.

Structurally, in some exemplary arrangements the face shield can be the supporting element on one, two, three or four sides for both the supply and exhaust air passages, and also for the air inlet and outlet openings.

In some exemplary arrangements the distance between the face shield and the face should be as small as possible, so as to make it difficult for ambient air to penetrate. At the same time, this freedom of breathing increases wearing comfort, which is particularly important for continuous use.

In order to adapt the distance between the inside of the face shield to varying face dimensions, a flexible seal which provides a closure of the remaining gap at the edge of the visor may be utilized in some arrangements. This can be done by adjusting or moving fixed elements of the face shield, or by using a cloth-like fabric that is able to close the air gap flexibly.

In some arrangements the face shield can be held around the head with fastening straps, by elastic bands, or attached to head coverings. In particular, protective helmets or baseball caps may be suitable, whereby a gap can be made in the upper end of the face shield, through which the peak can be inserted.

It is advantageous in some arrangements if the face shield, or the headpiece of the face shield, has a data recording device. For this purpose, the headpiece, and/or the face shield, may be provided with sensors and possibly an external power source (a power bank). However, it is also possible for the external power source to be used only to power the antibacterial and/or antiviral device and a fan. These sensors may detect operating and/or environmental conditions such as air pressure, flow velocity, movement, temperature and UVC light intensity, and alternatively or in addition motion sensors, distance sensors, a power supply sensor, etc., can be used for this purpose.

In addition, the processing of information from external data sources by circuitry of the headpiece of exemplary arrangements may also be carried out in some arrangements. For example, how many people in the environment are also wearing a face shield? By transmitting information (e.g. via the mobile phones of the people in the environment for example), the circuitry may determine and record or indicate to a user through an output device a different level of risk, depending on whether the people are immunized, vaccinated, or unvaccinated.

Since the exemplary headpiece is worn on the top of the user's head and against the forehead, it is also possible to include in the headpiece suitable sensors to record personal data measured on or via the skin on the head. This includes data such as temperature, heart rate, etc.

In some exemplary arrangements the face shield, or the headpiece of the face shield, may include circuity which includes a transmission and/or reception device. All current and future data transmission methods can be used. Both wired connections and wireless transmission paths (e.g. Bluetooth™) can be used between the components of the headpiece and other devices, such as external equipment, and/or the Internet.

In some exemplary arrangements the face shield, or the headpiece of the face shield, may include circuitry which operates as a data evaluation and or data storage device. In exemplary arrangements the data that is measured, determined, or transmitted may be stored in a data store or database, which can be saved in terms of extent and time depending on requirements, or can also be discarded after processing.

In exemplary arrangements depending on requirements, the stored data may be used on its own or in combination with appropriate algorithms. In some arrangements the stored data may be used for utilization and control of the headpiece itself, as well as for export and further processing.

Since the exemplary headpiece may be used in places where many people congregate, an appropriate enrichment of the air flow with fragrances can be a very positive, application-promoting additional benefit. For this purpose, in exemplary arrangements appropriate fragrance material and carriers can be integrated into the air flow. Odor neutralisation material may also be utilized. In exemplary arrangements the headpiece may include a source which serves as a repository of such material and a metering device with which to introduce the materials into the airflow.

To improve communication, in some arrangements a microphone and speakers is included in the apparatus. For example, the headpiece may have circuity that includes a communications device. In some arrangements the circuitry may provide for smartphones to be connected in such a way that calls can be made and received, either manually via a mobile phone, or via voice control utilizing the headpiece.

In some exemplary arrangements data from the headpiece is recorded continuously or at intervals, and the air supply is thereby controlled or regulated through operation of circuitry in the headpiece. In this way, operating conditions and performance data of the headpiece can be adapted to the current requirements. For example, someone sitting quietly alone, who is not exposed to any significant inflow of ambient air (with potential viral and/or bacterial contamination), requires a less disinfected supply of air than someone wearing the apparatus while walking through a supermarket, or while on a train, plane, bus, or in a lift. Circuitry of exemplary arrangements operates to change device operation to address different conditions and situations. Conclusions concerning effectiveness can be drawn from the data, including predications concerning the necessary lethal dose of determined UV radiation that is required for airborne diseases at a particular time.

For exemplary arrangements that serve as work equipment, personal protective equipment or medical devices, the documentation of the respective condition, operating data and/or effectiveness may be of great importance. In exemplary arrangements this can be done both by circuitry in the device itself, and, after transmission, in an appropriate app operating on a smartphone or other computer. In some arrangements headpiece data may be recorded and documented continuously, or at intervals.

The performance data of the headpiece of exemplary arrangements can be adjusted in accordance with predefined parameters, and the respective environmental data can be regulated in such a way that an optimum protective effect is achieved with maximum wearing comfort and maximum service life at the same time.

Advantageous examples of some exemplary arrangements and variants are shown in the Figures and are described in the following Detailed Description.

### BRIEF DESCRIPTION OF DRAWINGS

FIG. 1 is a schematic representation of an exemplary headpiece.

FIGS. 2 to 5 show an exemplary arrangement for use in a medical technology application.

### DETAILED DESCRIPTION

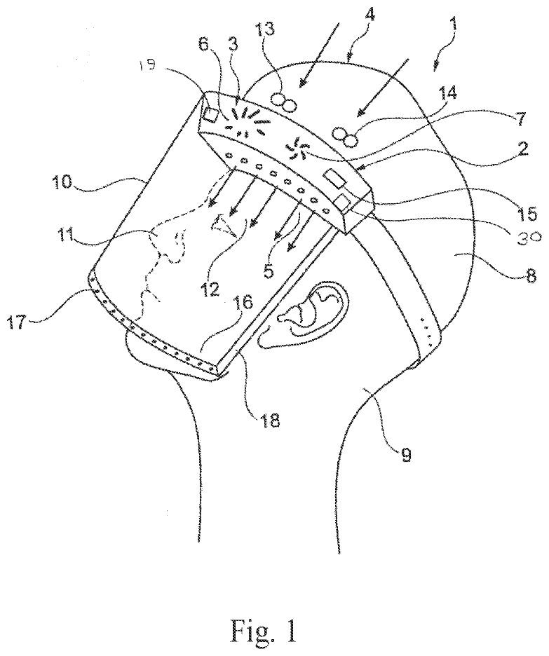

The exemplary headpiece 1 shown in FIG. 1 has a housing which serves as an air guide 2, and an antibacterial and/or antiviral device 3 which are alternatively referred to herein as disinfecting devices. The exemplary headpiece makes it possible to convey air from an inflow region 4 to an outflow region 5 with the air guide 2. The air passes through or adjacent to a plurality of disinfecting devices which may comprise UVC lamps 6, 7, by which the air is treated antibacterially and/or antivirally.

The exemplary headpiece 1 includes a head covering 8 with which it can be held on the head 9 of a person. A visor 10 on the exemplary headpiece 1 is positioned in front of the face 11 of the user's head 9 in such a way that air 12 exiting from the air guide through an outlet in the outflow region 5 adjacent the face 11 reaches the region of the mouth and nose of the user's face 11. One or a plurality of units generating a directed air flow are shown in the example arrangement as two fans 13, 14. These fans ensure that flowing air 12 passes adjacent the antibacterial and/or antiviral device 3 into the outflow area or region 5 between the user's head 9 and the inside surface visor 10. With the exemplary headpiece in place, air 12 thus flows from an inflow region 4 located above the outflow area of region 5, through the air guide adjacent the at least one antibacterial and/or antiviral device 3, through at least one outlet to the face 11.

In order to supply the UVC light sources 6, 7 with energy, the headpiece 1 has an energy source 15. This may be an energy source of the type previously discussed.

An exemplary headpiece thus essentially consists of the three main components: a mobile encapsulated UVC light source, a fan for directional forced ventilation, and, associated with these, at least one of a wide variety of head coverings that may be with and without face shields. A face shield is alternatively referred to herein as a visor.

The air 12, whether contaminated with or without viruses/bacteria, etc., is sucked in to the exemplary air guide via the fan 13, 14, led past the UVC light source 6, 7, and directed towards the user's face 11. As with an air shower, the face 11 and the inhaled air 12 are thus generally free of active viruses/bacteria, and other types of particles of airborne diseases.

In exemplary arrangements the air flow, the strength of the light source 6, 7, and the directed air flow are coordinated through control circuitry 19 in such a way that maximum purification is ensured with the lowest possible energy input.

These basic elements of the exemplary arrangement are completed with the energy source 15. Exemplary arrangements may include a variety of interchange and charging devices such as those which are used for other mobile devices, such as for charging mobile phones.

Optionally, in some arrangements a further outflow region or inflow region can be provided on one side. FIG. 1 shows an inflow region 16 with air exhaust openings 17 for drawing air from the space between the visor 10 and the face 11 via a passage 18, and also supplying it to the antibacterial and/or antiviral device 3, or another antibacterial and/or antiviral device.

The head coverings 8 that are integrated with the exemplary apparatus in some arrangements, may include a simple face shield, baseball caps, and hats, as well as head coverings for occupational safety such as helmets.

Exemplary arrangements may also include other components which have been previously discussed. Such components may include one or more lights, headlamps, cameras, microphones, filters, thermoelectric generators, thermal resonators, data recording devices, sensors for airflow rate, air velocity, air movement, air temperature and disinfectant light intensity. Such sensors may also include sensors for detecting motion, distance, the status of the power source, and/or may include sensors for detecting the temperature, heart rate or other conditions of the user wearing the apparatus. Exemplary arrangements may also include a repository or other sources of fragrance or odor neutralizer material. Exemplary arrangements may also include suitable circuitry that provide a wireless or wired communication device and/or data storage. Such components are represented individually and collectively in the exemplary arrangement schematically by component 30.

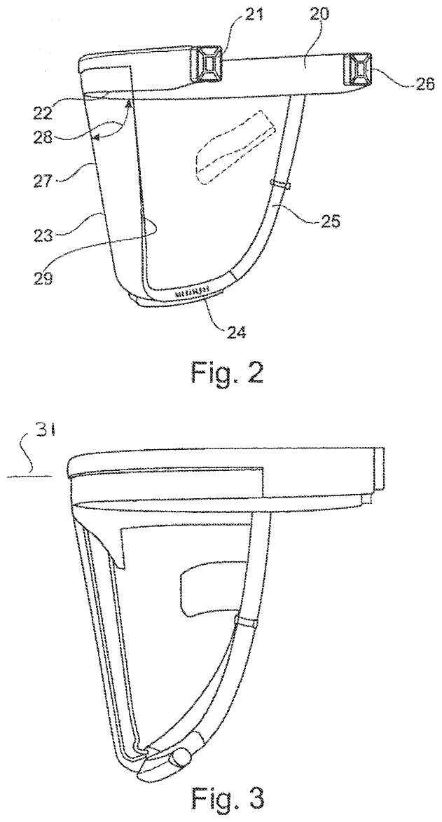

FIGS. 2 to 5 show an example arrangement of a two-sided utilization with supply and exhaust air. In this exemplary arrangement disinfected air can be supplied from above and below, but can also, for example, be supplied at the top and exhausted at the bottom. In exemplary arrangements combinations of the four edges may be utilized for air delivery or removal, together with a branch that directs disinfected air onto the surface of the face shield in front of the mouth/nose region. The constructional design of an exemplary arrangement is shown in the Figures.

FIG. 4 shows a headpiece that includes an exemplary headband 20 which serves as a housing and is curved into a U-shape, one side of which has an intake opening 21 for ambient air, through which air is sucked in. The headband bounds an opening in which the upper portion of the user's head is received as shown. The antibacterial and/or antiviral device (not shown) is arranged in the headband, with which the sucked-in air is disinfected by means of UV light. At least one outlet 22 is provided on the underside of the exemplary headband 20, through which disinfected air flows downwards behind the visor 23 and in front of a user's face. FIG. 5 shows that this air reaches the nose region of a person wearing the headband.

At the lower end of the exemplary visor 23 is at least one suction exhaust opening 24, through which exhaled air of the user is sucked in and directed via a hose 25 to the interior area of the headband. The negative pressure that causes the air to be sucked in through the suction opening 24 is generated in the headband. The exhaled air thus passes through the headband again, this time being passed outward through an exhaust opening 26 on the other side of the headband 20 and into the open air surroundings. The exhaled air can also be disinfected on its way from the suction opening 24 through the hose 25 and the headband, preferably in the interior air guide area of the headband 20.

The exemplary visor 23 has a front face 27 which is arranged at an angle 28 of about 85° to a central plane 31 of the headband 20. The two side faces 29 lie closer to the face and are therefore arranged at an angle of about 75° to the central plane of the U-shaped headband. The side faces of the exemplary visor join the front face of the visor in a curved shape, and at their ends opposite the front face they can still be curved slightly inwards towards the face, or have an end section that deflects the air towards the face.

The extraction of air from the interior area at the bottom of the visor 23 may not be done in some exemplary arrangements. Such an air extraction approach may be useful for certain applications such as medical applications, and work in a clean room.

In order to supply the headband with enough energy that is used for purposes of disinfection, the headband can be connected to a power bank.

Thus the exemplary arrangements achieve improved operation, eliminate difficulties encountered in the use of prior devices and methods, and attain useful results described herein.

In the foregoing description, certain terms have been used for brevity, clarity and understanding. However, no unnecessary limitations are to be implied therefrom because such terms are used for descriptive purposes and are intended to be broadly construed. Moreover the descriptions and illustrations herein are by way of examples and the new and useful concepts are not limited to the exact features and relationships that have been shown and described.

It should be understood that the features and/or relationships associated with one exemplary arrangement can be combined with features and/or relationships from another exemplary arrangement. That is, various features and/or relationships from various arrangements can be combined in further arrangements. The new and useful scope of the disclosure is not limited only to the exact arrangements that have been shown and described herein.

Having described features, discoveries and principles of the exemplary arrangements, the manner in which they are constructed and operated, and the advantages and useful results attained, the new and useful features, devices, elements, arrangements, parts, combinations, systems, equipment, operations, methods, processes and relationships are set forth in the appended claims.

1.-29. (canceled)

30. Apparatus comprising:
a headpiece configured to be worn in supported relation with a head of a user of the apparatus, the wherein headpiece includes
a U-shape headband, wherein the headband is configured to receive the head of the user within the headband,
an air guide integral with the headband, wherein the air guide is configured to enable air flow through the air guide,
at least one disinfecting device, wherein the at least one disinfecting device is housed within the air guide and is operative to kill airborne disease particles in air passing through the air guide,
wherein the air guide is configured to deliver disinfected air through at least one air guide outlet to the user.

31. The apparatus according to claim 30, and further comprising:
a head cover, wherein the head cover is configured to extend over the top of the head of the user.

32. The apparatus according to claim 30 and further comprising:
a visor, wherein in operative position of the apparatus the visor extends in front of a face of the user,
wherein the at least one air guide outlet is operative to deliver air into an area intermediate of the face of the user and the visor.

33. The apparatus according to claim 30 and further comprising:
a visor, wherein the visor includes
a visor front, wherein in an operative position of the apparatus the visor front extends in front of a face of the user, and
a pair of visor sides, wherein in the operative position of the apparatus each respective visor side extends on a respective side of the face of the user,
wherein the visor front extends at a visor front angle relative to a central plane of the headband, and each visor side extends at a visor side angle relative to the central plane of the headband,
wherein the visor front angle is greater than the visor side angle.

34. The apparatus according to claim 30 and further comprising:
a visor, wherein the visor includes
a visor front, wherein in an operative position of the apparatus the visor front extends in front of the face of the user,
wherein the visor front extends at an angle of about 85° to a central plane of the headband.

35. The apparatus according to claim 30 and further comprising:
a visor, wherein the visor includes
a visor front, wherein in an operative position of the apparatus the visor front extends in front of a face of the user,
a pair of visor sides, wherein in the operative position of the apparatus each respective visor side extends on a respective side of the face of the user,
wherein each visor side extends at an angle of about 75° to a central plane of the headband.

36. The apparatus according to claim 30 and further comprising:
a visor, wherein in the operative position of the apparatus the visor extends in front of a face of the user,
wherein the at least one air guide outlet is intermediate of the face of the user and the visor, and is positioned on at least one transverse side of the face of the user.

37. The apparatus according to claim 30 and further comprising:
a visor, wherein in an operative position of the apparatus the visor extends in front of a face of the user of the apparatus, wherein the at least one air guide outlet is positioned intermediate of the face of the user and the visor,
an air exhaust opening, wherein the air exhaust opening extends intermediate of the face of the user and the visor, wherein the air exhaust opening is connected to open air.

38. The apparatus according to claim 30 and further comprising:
a visor, wherein in an operative position of the apparatus the visor extends in front of a face of the user of the apparatus, wherein the at least one air guide outlet is positioned to deliver air intermediate of the face of the user and the visor,
an air exhaust opening, wherein the air exhaust opening is positioned intermediate of the face of the user and the visor, wherein the air exhaust opening is connected to the air guide through an exhaust passage,
wherein air exhausted from the exhaust opening is passed through the air guide and is disinfected by the at least one disinfecting device prior to being delivered from the air guide to open air.

39. The apparatus according to claim 30
wherein the headpiece includes an air intake and an air outlet, wherein the air intake is disposed on the headpiece away from the air outlet,
wherein air drawn in through the air intake is delivered into the air guide,
and further comprising:
a visor, wherein in an operative position of the apparatus the visor extends in front of a face of the user of the apparatus, wherein the at least one air guide outlet is positioned to deliver air intermediate of the face of the user and the visor,
an air exhaust opening, wherein the air exhaust opening is positioned intermediate of the face of the user and the visor, wherein the air exhaust opening is connected to the air guide through an exhaust passage,
wherein air from the exhaust opening is passed through the air guide, is disinfected by the at least one disinfecting device and is then delivered from the air outlet.

40. The apparatus according to claim 30, and further comprising:
a visor, wherein in an operative position of the apparatus the visor extends in front of a face of the user,
wherein the at least one air guide outlet is configured to deliver air intermediate of the face of the user and the visor,
wherein the visor includes at least one edge, wherein in the operative position a flexible seal extends between the at least one edge of the visor and the face of the user.

41. The apparatus according to claim 30, and further comprising:
at least one fan, wherein the at least one fan is operative to cause air to move through the air guide and to the at least one air guide outlet.

42. The apparatus according to claim 30 wherein the at least one disinfecting device includes at least one of
a UV lamp,
a UVC light source,
a filter, and
an electrostatic filter.

43. The apparatus according to claim 30 and further comprising:
at least one of a light, a camera, a microphone and a visual aid in supported connection with the headband.

44. The apparatus according to claim 30 and further comprising:
at least one source of fragrance or odor neutralizer material in operative supported connection with the headband.

45. The apparatus according to claim 30 and further comprising:
at least one sensor in operative supported connection with the headband,
circuitry including at least one data store in operative supported connection with the headband, and
at least one communication device in operative supported connection with the headband.

46. The apparatus according to claim 30 and further comprising:
at least one sensor in operative supported connection with the headband,
wherein the at least one sensor is operative to sense at least one of
air velocity,
air temperature,
an operational characteristic of the at least one disinfecting device,
motion,
distance,
user temperature, and
user heart rate,
circuitry including at least one data store in operative supported connection with the headband, wherein the circuitry is operative to store in the at least one data store, data corresponding to data sensed by the at least one sensor,
at least one communication device in operative supported connection with the headband, wherein the at least one communication device is operative to communicate data stored in the at least one data store from the apparatus.

47. The apparatus according to claim 30 and further comprising:
circuitry in operative supported connection with the headband,
at least one of a thermoelectric generator and a thermal resonator in operative supported connection with the headband, wherein the thermoelectric generator is operative to supply electrical power to the circuitry.

48. Apparatus comprising:
a headpiece configured to be worn in supported relation with the head of the user of the apparatus, including
a headband, wherein the headband is configured to receive an upper portion of the head of the user within an opening thereof,
a housing integral with the headband, wherein the housing bounds an interior area configured to enable air to pass therethrough,
a fan in operative connection with the housing, wherein the fan is operative to cause air to move through the housing to at least one housing outlet,
at least one disinfecting device in operative connection with the housing, wherein the at least one disinfecting device is operative to prevent live airborne disease particles from passing through the housing to the at least one housing outlet,
a visor in operatively attached connection with the headband, wherein in an operative position of the apparatus the visor extends in outwardly overlying relation of a face of the user,
wherein the at least one housing outlet is fluidly connected to a space intermediate of the face
of the user and the visor.

49. Apparatus comprising:
a headpiece configured to be worn in supported relation with the head of a user of the apparatus, wherein the headpiece includes
a head opening, wherein the head opening is configured to receive an upper portion of the head of the user therein,
an air intake configured to receive air, and an air outlet configured to exhaust air,
a housing in operative supported connection with the headpiece, wherein the housing includes an interior area configured to enable air to flow therethrough,
at least one fan, wherein the at least one fan is configured to cause air to be drawn into the air intake and to cause air to be exhausted from the air outlet,
at least one air purifier, wherein the at least one air purifier, is operative to prevent live airborne disease particles from passing through the interior area in air that passes through in the interior area of the housing,
a visor, wherein the visor is configured such that in the operative position of the apparatus the visor is positioned in overlying relation of a face of the user and bounds an area between the visor and the face of the user,
wherein the fan is operative to cause the air drawn into the air intake to be passed through the housing interior area and acted on by the at least one air purifier to produce purified air, and the purified air to be delivered to the area between the visor and the face of the user,
wherein air in the area between the visor and the face of the user is caused to be drawn out of the area between the visor and the face of the user, passed through the interior area and acted on by the at least one air purifier to produce further purified air, and the further purified air exhausted through the air outlet.
